(19) United States
(12) Patent Application Publication
(10) Pub. No.: US 2004/0053342 A1
(43) Pub. Date: Mar. 18, 2004

(54) Measurement of analytes

(76) Inventors: Alexander D. Romaschin, Etobicoke (CA); Paul M. Walker, Toronto (CA)

Correspondence Address: KLAUBER & JACKSON, 411 HACKENSACK AVENUE, HACKENSACK, NJ, 07601

(21) Appl. No.: 10/614,678

(22) Filed: Jul. 7, 2003

Related U.S. Application Data
(63) Continuation-in-part of application No. 09/585,582, filed on Jun. 2, 2000, now abandoned.
Application No. 09/585,582 is a continuation-in-part of application No. 09/353,189, filed on Jul. 14, 1999, now Pat. No. 6,306,614.
Application No. 09/585,582 is a continuation-in-part of application No. 09/457,465, filed on Dec. 8, 1999, now Pat. No. 6,203,997.
Application No. 09/457,465 is a continuation of application No. 08/991,230, filed on Dec. 16, 1997, now abandoned.
Application No. 08/991,230 is a continuation-in-part of application No. 08/552,145, filed on Nov. 2, 1995, now Pat. No. 5,804,370.
Application No. 08/552,145 is a continuation-in-part of application No. 08/516,204, filed on Aug. 17, 1995, now abandoned.
Application No. 08/516,204 is a continuation-in-part of application No. 08/257,627, filed on Jun. 8, 1994, now abandoned.

(51) Int. Cl. G01N 33/53; G01N 33/567; C12N 15/09

(52) U.S. Cl. 435/7.2

(57) ABSTRACT

The invention relates to a method for measuring the level of a preselected analyte in a sample such as of blood of a human or animal patient by incubating the test sample with an antibody specific to the analyte to form an immunocomplex, which then interacts with the white blood cells present in or added to the sample and result in the production of oxidants. Oxidants are detected using chemiluminescent reagents. The assay is performed on the sample and in addition may include a measurement of the oxidant production resulting from a maximal stimulatory dose of immunocomplexes, providing a ratio to indicate the level of analyte in the sample. The white blood cell oxidant response may be enhanced by the inclusion of certain agents such as zymosan or complement. This method may be used to determine levels of analytes in a sample of a patient's blood including endotoxin and other analytes related to sepsis, in order to select the proper therapeutic course, or may be used to measure other analytes such as inflammatory mediators, hormones, acute phase proteins, toxins, drugs of abuse, markers of cardiac muscle damage, therapeutic drugs, cytokines, and chemokines.

### CROSS-REFERENCE TO RELATED APPLICATIONS

This Application is a Continuation in Part of:

i) U.S. patent application Ser. No. 09/585,582 which is a continuation-in-part of application Ser. No. 09/353,189, filed Jul. 14, 1999; and a continuation-in-part of Ser. No. 09/457,465, filed Dec. 8, 1999, which is a continuation of Ser. No. 08/991,230, filed Dec. 16, 1997, now abandoned; both of which are a continuations-in-part of Ser. No. 08/552,145, filed Nov. 2, 1995; now U.S. Pat. No. 5,804,370; which is a continuation-in-part of Ser. No. 08/516,204, filed Aug. 17, 1995, abandoned; which is a continuation-in-part of Ser. No. 08/257,627, filed Jun. 8, 1994, abandoned; and

ii) U.S. patent application Ser. No. 09/961,889, which is a continuation-in-part of application Ser. No. 08/552,145, filed Nov. 2, 1995, now U.S. Pat. No. 5,804,370, which is a continuation-in-part of application Ser. No. 08/516,204, filed Aug. 17, 1995, abandoned, which is a continuation of application Ser. No. 08/257,627, filed Jun. 8, 1994, abandoned. All of the foregoing applications are incorporated herein by reference in their entireties.

### BACKGROUND OF THE INVENTION

Rapid quantitation of specific analytes that may be present in a bodily fluid such as whole blood is critically important for the diagnosis of disease and its severity, often under emergency conditions, in the monitoring of the progression of pathological conditions and following the recovery process brought about by surgical and drug therapies. It is often important to know not only whether a specific analyte is present, but also its level, in order to determine the present stage of a particular condition or disease and in order to prescribe the most effective remedy at that particular stage. In the treatment of many diseases, a particular therapy may be ineffective or toxic if given at the wrong stage of the condition. For example, the levels of specific markers of cardiac muscle damage and the relationship among them may indicate that a patient has had or may be having a heart attack.

The level of a therapeutic drug in the circulation may indicate whether the patient is being dosed optimally, and whether presumptive side effects are possibly due to excess levels of the drug. In infection and sepsis, the circulating levels of infectious microorganism-derived toxins and inflammatory mediators produced by the patient's white blood cells in response to these toxins may indicate the severity and level or stage of sepsis and help identify the most efficacious course of therapy. Quantitation of analytes under emergency conditions and using the information to prescribe a particular therapy may mean the difference between saving a patient's life and contributing to the patient's death.

For example, in the case of infection, hospital and particularly intensive care unit patients who have acquired nosocomial infections as a result of peri- or post-operative immunosuppression or infections secondary to other disease processes, such as pancreatitis, hypotensive or hypovolemic shock, physical trauma, burn injury, or organ transplantation, and subsequently develop septic shock syndrome, have a mortality which has been quoted to range from 30-70% depending upon other co-incident complications. Despite the development of increasingly potent antimicrobial agents, the incidence of nosocomial infections and, in particular, infections leading to sepsis or septicemia, is increasing. The difficulty with many of the promising therapeutic agents is that their window of opportunity and indications for use have not been adequately delineated largely due to a lack of appropriate rapid and quantitative diagnostic procedures and partly due to a lack of complete understanding of the pathogenesis of the sepsis syndrome.

The presence of bacteria, viruses or fungi or their cell wall components including gram- positive peptidoglycans, lipoteichoic and teichoic acids, and gram-negative endotoxin (lipopolysaccharide, LPS) in blood is indicative of an infection. In addition, the immune system's reaction to the presence of these foreign antigens by the production of pro-inflammatory cytokine mediators such as interleukin-1 (IL-1), tumor necrosis factor (TNF) and interleukin-6 (IL-6), is also indicative of an infection. The quantity of these analytes in circulation may be used to indicate the severity and level or stage of sepsis. For instance, at an early stage of Gram-negative sepsis, LPS may be present at a concentration as low as 50 pg/ml of whole blood. At the next stage, sepsis has progressed and a mediator of sepsis, TNF, can be detected and measured using antibody against TNF. At stage 3, TNF may be present in smaller amounts since it is transitory and another transitory mediator, IL-1, may appear. As sepsis progresses further, LPS levels may decrease and TNF may be absent, but IL-1 may increase and interleukin-6 (IL-6) may appear. Finally, in a more prolonged case of sepsis, LPS may be present and IL-1 may be at low levels but IL-6 may be at very high levels. Thus, diagnosis of sepsis and identification of its stage in the course the disease are critical for the successful treatment of this serious and potentially lethal consequence of infection. Quantitation of the levels of the sepsis-associated analytes provide information necessary to determine the best course of therapy to treat the acute disease.

Currently, one of the major problems with many of the therapeutic protocols being tested by the pharmaceutical companies conducting clinical trials in sepsis intervention is their inability to rapidly detect early and evolving sepsis. The results of blood cultures may arrive too late. Other septicaemia tests are also time consuming and may not be sensitive enough for early detection. Centocor Inc.'s immunometric assay for tumor necrosis factor-alpha (TNF-α), as described in WO 90/06314, uses two antibodies, one of which is labeled. The National Aeronautics and Space Administration detects Pseudomonas bacteria by extraction of Azurin and detection using Azurin-specific antibody (U.S. Pat. No. 7,501,908). The endotoxin assay kit from BioWhittaker (Walkerville, Md., U.S.A.) or Seikagaku Kogyo Ltd. (Tokyo, Japan) is a Limulus Amebocyte Lysate (LAL) Assay technique which may be used as a comparison for the present invention.

Many investigators versed in the complexities of the septic response believe that treatment is ineffectual for patients who already manifest the classical clinical symptoms of sepsis (i.e., hyperdynamic circulation, hypotension, decreased systemic vascular resistance, pyrexia and increased oxygen dependency). The course of the inflammatory process has progressed too far for many of the interventions to benefit the patient since the multiple interacting inflammatory cascades with which the body attempts to eliminate the infectious challenge are in many instances at their nadir and difficult to control pharmacologically. Thus, a major clinical and diagnostic challenge is to identify and stage patients, ideally early in the progression of the septic response, or to identity those patients at high risk of developing fulminant sepsis syndrome. The same therapeutic agents given at the one stage in the septic process may have more significant beneficial effects than when given at another, since it is clear that an optimal window period may exist for the efficacy of any particular therapeutic agent. For example, giving a patient antibodies or receptors directed against gram-negative endotoxins when the patient has no detectable levels of these agents present in the circulation and already has a maximally activated cytokine cascade is a waste of resources and of no benefit to the therapy of the patient. The potential market for these anti-sepsis strategies remains large (about 250,000 cases per year in the USA) and has been limited by the inability to identify and stage patients who could benefit from the appropriate pharmacologic interventions.

It is toward the development of improved methods for the rapid quantitation of analytes in a bodily fluid sample such as a whole blood sample, that the present application is directed.

### SUMMARY OF THE INVENTION

In its broadest aspect, the present invention is directed to a method for detecting an analyte in a sample which comprises:

(a) forming an immunological complex between the analyte and an antibody thereto;

(b) reacting the immunological complex with an oxidant-producing phagocytic cell or extract thereof; and

(c) measuring the amount of oxidant produced by the oxidant-producing phagocytic cell as an indicator of the presence or absence of said analyte in said sample.

The analyte is any substance or component such as may be present in a bodily fluid sample which can participate in the formation of an antigen-antibody complex (immunocomplex or immunological complex) with added, exogenous antibody. For example, analytes may include gram-positive bacteria, gram-negative bacteria, fungi, viruses, gram-positive cell wall constituents, lipoteichoic acid, peptidoglycan, teichoic acid, gram-negative endotoxin, lipid A, hepatitis A, inflammatory mediators, drugs of abuse, therapeutic drugs, or cardiac markers, such as myoglobin, creatine kinase MB, troponin I or troponin T. Inflammatory mediators include but are not limited to tumor necrosis factor, interleukin-1, interleukin-6, interleukin-8, interferon, and transforming growth factor P. The analyte may be one indicative of infection or indicative of sepsis.

Examples of samples which are bodily fluids that are useful in the practice of the invention include, but are not limited to, whole blood, plasma, serum, urine, saliva, and cerebrospinal fluid.

The antibody may be, for example, a monoclonal antibody, a polyclonal antibody, a chimeric antibody, and any combination of such antibodies. The monoclonal antibody may be an IgM, an IgG or an IgA. Other immunoglobulins whose immunocomplexes are capable of generating oxidants from white blood cells may also be used.

The oxidant-producing phagocytic cells may be those already present in a biological sample such as a bodily fluid, or oxidant-producing phagocytic cells from an exogenous source may be added to the sample. Preferably, oxidant-producing phagocytic cells are present in the sample, in particular a biological sample and more preferably a biological sample such as a bodily fluid including but not limited to whole blood. In such cases no addition of exogenous oxidant-producing cells is necessary. Useful endogenous or added cells include but are not limited to neutrophils, lymphocytes, monocytes, and any combination thereof. Added cells may be derived from tissue culture, immortalized white cell cultures, such as HL-60, enucleated cells, or artificially-prepared vesicles comprising the machinery to generate oxidants.

In addition to the steps recited above, various activators and other components may be added to enhance the production of oxidants by the phagocytic cells in the presence of immunocomplexes. For example, an activator of oxidant production optionally may be included in step (b) of the hereinabove method to enhance the production of oxidants. Non-limiting examples of such activators include zymosan, opsonized zymosan, latex beads and opsonized latex beads. Other agents useful for this purpose include a phorbol ester or N-formyl-met-leu-phe. In addition, complement proteins may be included in step (b) to enhance the oxidant production from immunocomplexes present in the sample. Such complement may be present endogenously if the sample is a bodily fluid, or complement proteins may be added to the assay. Complement or complement proteins as used herein refers to one or more complement proteins or factors naturally present in plasma that enhance oxidant production by white blood cells.

The method used for measuring the amount of oxidant produced by the phagocytic cells maybe measured by methods such as chemiluminescence, measurement of change in redox potential by electrochemical probe, oxidation of a chromogenic or fluorogenic substrate, and the like. Chemiluminescence is preferred. When chemiluminescence is measured, a chemiluminescent compound such as, but not limited to, luminol, lucigenin and pholasin is included in step (b) of the method described hereinabove. The other methods for measuring oxidant production have their corresponding appropriate reagents. An instrument for measuring the readout of the oxidant production, such as a luminometer or scintillation counter for chemiluminescence, a spectrophotometer or fluorimeter for chromo- or fluorogenic substrates, or the associated electronics with a redox probe, may also be used to record and display the generation of oxidants. Preferably, the instrument integrates the oxidant output of each tube over time, and additionally, may be programmed to perform the calculations as described herein to readout the results, such as in the case of an analyte from a bodily fluid, level of sepsis-related analyte. The present invention may also be adapted to a test strip format, for ease in use at the bedside or other locations where assay componentry may be lacking, such as in the field, for example in testing water contamination, and where a qualitative yes/no readout may be sufficient rather than a quantitative result, utilizing with a calorimetric readout. A yes/no readout may also be appropriate for certain medical uses, such as indicating if a particular sepsis-related antigen or cytokine is present at a level above a certain critical level, providing a “yes” or “no” answer to rule in or rule out sepsis, for example. Various other configurations are embraced for the present methods.

Various modifications of steps (a) through (c) may be carried out in alternate embodiments of the present invention, for example, to include a control or controls to increase the accuracy or quantitative aspect of the method. For example, a control assay may be carried out in parallel with the described steps using an antibody of the same class but not directed to the particular analyte being measured. In another embodiment, dilutions of the antibody may be provided to offer various levels of detectability of the analyte, to offer a semi-quantitative assay. In yet another embodiment, a one-point calibrator in the form of a test and its control for the maximum responsiveness of white blood cells in the sample to immunocomplexes may be provided, from which the readout for the analyte being measured may be compared to provide a quantitative output. The maximum responsiveness may be determined by providing a maximal amount of immuncomplexes of the same analyte as being measured, by providing authentic analyte and using the antibody to the analyte in a separate determination, or an unrelated, second analyte and an antibody thereto. In yet still another embodiment, a single test for the maximum responsiveness of white blood cells may be provided without a corresponding control, to offer a test that reads out in relative units of the analyte, useful for determining to what extent the patient's analyte level is above or below a critical level. These and other variations on the broadest aspect of the invention are fully embraced herein.

As mentioned above, analytes that may be detected by a method of the present invention may include gram-positive bacteria, gram-negative bacteria, fungi, viruses, gram-positive cell wall constituents, lipoteichoic acid, peptidoglycan, teichoic acid, gram-negative endotoxin, lipid A, hepatitis A, inflammatory mediators, drugs of abuse, therapeutic drugs, or cardiac markers, such as myoglobin, creatine kinase MB, troponin I or troponin T. In a preferred embodiment, the analyte is indicative of sepsis or infection and may be, by way of example, Gram-positive bacteria, Gram-negative bacteria, fungi, viruses, protists, Gram-positive cell wall constituents, Gram-negative endotoxin (lipopolysaccharide), lipid A, and inflammatory mediators. Non-limiting examples of Gram-positive bacteria include Staphylococcus aureus, Enterococcus faecalis, Streptococcus. pyogenes, Listeria monocytogenes, Streptococcus sanguis, Streptococcus pneumoniae, Staphylococcus epidermitis, and Bacillus subtilis. Gram-negative bacteria include but are not limited to Escherichia coli, Shigella flexneri, Pseudomonas aeruginosa, Salmonella Minnesota, and Klebsiella pneumoniae. Non-limiting examples of fungi include Candida albicans, Aspergillis flavus, Histoplasma capsulatum, Coccidioides immitis, and Cryptococcus neoformans. Examples of viruses include but are not limited to hepatitis A, herpes simplex viruses 1 and 2, hepatitis B, influenza virus, and human immunodeficiency virus. Protistan species include but are not limited to Cryptosporidium parvum. The aforementioned Gram-positive cell wall constituents include, but are not limited to, lipoteichoic acid, peptidoglycan, teichoic acid, and M protein. Non-limiting examples of inflammatory mediators include tumor necrosis factor, interleukin-1, interleukin-6, interleukin-8, interferon and transforming growth factor β.

The foregoing analytes are merely exemplary of the invention and are non limiting; the invention may be used to detect any analyte for which an immunocomplex of the analyte with an antibody thereto may be formed and induce oxidant production by phagocytic cells, as described hereinabove.

The invention is also directed to a kit comprising componentry enabling the carrying out of the aforementioned assay and containing one or more of the aforementioned reagents. By way of non-limiting example, a first container may be provided of IgM, IgG or IgA antibody specific to the preselected analyte; and a second container of chemiluminescent compound. The chemiluminescent compound may be luminol, lucigenin or pholasin. In another embodiment, the aforementioned components may be provided in a single container. In another embodiment wherein a single-point calibrator of a maximal immunostimulatory amount of immunocomplexes is used, the aforementioned kit may further include a third container of analyte. The analyte for determining the responsiveness to the maximal immunostimulatory amount of immunocomplexes may be the same preselected analyte or a second, unrelated analyte; in the latter case an additional antibody to the second analyte must be provided. A source of oxidant-producing phagocytic cells may be included in the kit for samples which do not contain an adequate amount; the cells may be neutrophils, lymphocytes, monocytes, or combinations thereof. The kit may also include an additional container containing, or the single container may further contain, an agent capable of increasing oxidant production by white blood cells on exposure to immunocomplexes, for example, zymosan, latex particles, opsonized zymosan, opsonized latex particles, a phorbol ester, N-formyl-met-leu-phe, or combinations. A further component of the kit can be complement proteins.

These and other aspects of the invention will be appreciated from the following brief description of the drawings and detailed description of the invention.

### BRIEF DESCRIPTION OF THE DRAWINGS

FIG. 1 is a graph illustrating the chemiluminescent response of whole blood with monoclonal antibody and with 100 pg/ml endotoxin and without endotoxin.

FIG. 2A is a draft illustrating the chemiluminescent response using blood from a patient with severe sepsis syndrome who died 6 hours after the sample was taken, as compared to a control antibody of the same class, isotype and concentration but directed against irrelevant epitopes.

FIG. 2B uses blood from a healthy ambulatory volunteer.

FIG. 2C uses blood from a patient with chronic sepsis.

FIG. 2D uses blood from a patient with severe sepsis syndrome which contributed to his death 3 days after the sample was taken. This patient had no evidence of Gram-negative endotoxemia or bacteremia.

FIG. 2E uses blood from a patient being weaned from respiratory support and seriously cachectic, but with no clinical evidence of any septic foci.

FIG. 3 is the chemiluminescent response using blood from a patient with a leaky duodenal ulcer.

FIG. 4 shows results from the whole blood chemiluminescence response using varying concentrations of endotoxin with a fixed concentration of antibody against the endotoxin. Results are shown in linear form.

FIG. 5 shows results from the whole blood chemiluminescence response using varying concentrations of endotoxin with a fixed concentration of antibody against the endotoxin. Results are shown in logarithmic form.

FIG. 6 is a typical, whole-blood chemiluminescence profile of a patient with endotoxemia. Curve A represents whole blood plus zymosan; B, whole blood plus zymosan plus anti-endotoxin antibody; C, whole blood plus zymosan plus exogenous endotoxin (800 pg/ml); and D, whole blood plus zymosan plus exogenous endotoxin (800 pg/ml) plus anti-endotoxin antibody.

FIG. 7 demonstrates a dose response of endotoxin (“LPS”) versus response factor (RF), calculated as ∫(B-A)/∫(D-C), where the values A B, C, and D represent 15 minute reaction integrals of the chemiluminescence of the samples depicted in FIG. 8.

FIG. 8 compares (A) the Limulus amoebocyte assay (LAL) endotoxin assay to (B) the method described in the present invention.

FIG. 9 depicts a typical, whole-blood chemiluminescence profile of a sample from a patient with endotoxemia. Curve A represents whole blood plus zymosan; B, whole blood plus zymosan plus anti-endotoxin antibody; and C, whole blood plus zymosan plus anti-endotoxin antibody plus exogenous endotoxin (800 pg/ml).

### DETAILED DESCRIPTION OF THE INVENTION

Definitions

“Analyte” is defined as the specific substance of interest present in a sample such as a bodily fluid sample and being analyzed by the methods of the present invention. In the case of analytes related to infection and sepsis, these may include, for example, microorganisms and their components, including gram positive cell wall constituents and gram negative endotoxin, lipopolysaccharide, lipoteichoic acid, and the inflammatory mediators that appear in circulation as a result of the presence of these components, including tumor necrosis factor (TNF), interleukin-1 (IL-1) and other interleukins and cytokines. Other analytes may include drugs of abuse, hormones, toxins, therapeutic drugs, markers of cardiac muscle damage, etc.

“Sepsis” is defined as a pathological condition of the body resulting from the presence of infectious microorganisms, which clinically manifests as one or more of the following sequelae: pyrexia, hypotension, hypoxemia, tachycardia, hypothermia, neutrophilia, and neutropenia.

“Immunocomplexes” is a synonym for antigen-antibody complexes.

“Opsonized” refers to a particle to which immunoglobulin and complement factors are bound and which results in a more vigorous recognition of the particle by the immune system. For example, the yeast polysaccharide zymosan, or latex particles, may be opsonized by binding of immunoglobulin and complement factors to their surfaces; opsonized zymosan or latex will stimulate increased oxidant production by white cells after they are activated by exposure to immunocomplexes.

“Responsiveness” is a measure of the patient's ability to respond to a maximum stimulatory dose of immunocomplex.

The invention herein is broadly directed to a general method for determining the presence or level of an analyte in a sample by relying on oxidant-producing phagocytic cells present in and/or added to the sample to generate oxidants in proportion to the level of immunocomplexes formed from the analyte and anti-analyte antibody added to the sample. Oxidant level may be measured by any of several methods; chemiluminescence is preferred. Several variations and embodiments of the method are described herein, including qualitative, semi-quantitative, and quantitative procedures. These procedures optionally may utilize controls or other tests run concurrently or successively with the assay to provide the necessary information for interpreting the results of the test. The method generally comprises:

i) incubating the sample with an amount of antibodies specific to the analyte to form antibody/antigen complexes (immunocomplexes) therewith;

ii) allowing the immunocomplexes to interact with white blood cells or cell fractions or extracts thereof which results in the production of oxidants;

iii) measuring the amount of oxidant produced as a measure of the level of the analyte in the sample.

In a preferred embodiment of the invention, whether the procedure provides a qualitative, semi-quantitative, or quantitative readout of analyte level, the level of oxidants may be measured using a chemiluminescent compound which generates light in proportion to the amount of oxidants in the sample. Thus, in this preferred embodiment, a chemiluminescent compound is introduced into the steps above, allowing the oxidants to react with the chemiluminescent compounds to emit luminescent light from the test sample. Subsequently, emitted light is measured over a predetermined period, and correlated with the level of the analyte. In certain embodiments of the invention, the light output is measured by comparison of the measured amount of emitted light of the test sample with a measured amount of light emitted by a control sample which is treated in a similar fashion as the test sample except that control antibodies are used which are of the same class as the test antibodies but are non-specific to the analyte. Other controls may be used to reduce background values, or allow the assay to be performed in a semi-quantitative or quantitative fashion. In one embodiment, a concurrent measurement of the chemiluminescent response to a maximal amount of immunocomplexes may be measured by including adding a specific amount of authentic preselected analyte to the aforementioned components, and this single measurement, with or without a corresponding control therefor, used to render the test results quantitative or semiquantitative. Alternatively, this maximal responsiveness of the phagocytic cells and activators in the assay may be obtained from an analyte unrelated to the preselected analyte, by providing a second analyte and an antibody thereto. Preferably, the preselected analyte is the same as the second analyte, as the same antibody is used. These and other embodiments will be described in more detail below.

The general method described above may be performed with an activator of oxidant production, such as but not limited to zymosan, opsonized zymosan, latex beads and opsonized latex beads. Other agents useful for this purpose include a phorbol ester or N-formyl-met-leu-phe (FMLP). In addition, complement factors or proteins may be included as an activator to increase oxidant output by the phagocytic cells; if a bodily fluid is the source of sample, adequate complement factors may be present therein to suffice as an activator without requiring any additionally. The method may also include an exogenous source of oxidant-generating cells or acellular entities useful for the same purpose, particularly when the sample has no or insufficient levels of such cells to generate the measurable response. All of the methods described herein may have such components optionally included to enhance the detection.

Although the ensuing discussion relates to medically-related diagnostic applications of the aforementioned methods and preferably where the sample is a bodily fluid sample and the sample taken from the patient contains phagocytic cells and other components such as complement, the invention is not so limiting and one of skill in the art will readily adapt the assay to measure any analyte for which an immunocomplex therewith may be formed.

Various embodiments of the invention herein may be employed, depending on the level of sensitivity desired for the assay. As mentioned above, the assay may be qualitative, to give a “yes” (i.e., rule in) or “no” (i.e., rule out) answer, wherein a “yes” indicates a level of analyte above a predetermined value which is diagnostically indicative; a “low,” “intermediate,” or “high” level of analyte; or a quantitative value for the level of the analyte in the sample. Other types of assay readouts are also possible and are embraced herein.

For example, and as described in U.S. Pat. No. 5,804,370, incorporated herein by reference in its entirety, a two-tube assay may be performed to provide a qualitative measure of the presence of infection or sepsis analytes, and further may be applied to the detection of other analytes such as but not limited to those described herein. As noted herein, one tube comprises the antibodies to the suspected analyte, and another tube is identical except that the antibodies to the analyte are replaced with antibodies of the same class directed to an irrelevant antigen. The difference in chemiluminescence is determined, and an elevation over the value of the control indicates the presence of analyte. Some quantitation of the result is possible to provide an indication of level of analyte, as described below.

In a semi-quantitative version of the two-tube assay, the assay is performed in triplicate with different dilutions of the antibodies, e.g., 1:10, 1:100 and 1:1,000. A maximal signal is generated only at a particular ratio of antibody to antigen wherein maximum complementarity of the antibody and analyte in the sample produces the maximal amount of immunocomplexes. The readings of the three dilutions give a reading of the relative amount of analyte present. For example, at low analyte levels, only the 1:1,000 dilution will be positive; at a higher level, both the 1:1000 and 1:100 will be positive; at an even higher level, all three will be positive. As noted below, such discrimination can provide, for example, a value of below 20 pg/ml endotoxin, 20-100 pg/ml, or greater than 100 pg/ml.

In further, quantitative embodiments of the invention herein, it was found that by utilizing a type of control in which white cells in the sample, or those added thereto, are maximally stimulated by immunocomplexes, the correlation between the chemiluminescence of a sample and this maximum chemiluminescent output follows a predictable relationship and can be used to interpolate analyte levels. The maximum stimulatory dose of immunocomplexes may comprise the same analyte as is being quantitated, or may be another antigen (i.e., a second analyte) and its corresponding antibody. As used herein, the term “analyte” or “preselected analyte” refers to the substance being qualitatively or quantitatively measured such as a sepsis- or infection-related substance, by way of example, and the term “antigen” or “second analyte” is used to refer to the that same authentic substance or another substance that is provided along with the antibody to the substance already used in the assay, or alternatively to the second analyte, to maximally stimulate white blood cells present in the sample. For convenience, and particularly in the 3-tube assay described below, a preferred embodiment is where the antigen is the same as the analyte: for example, if endotoxin is the analyte, the control immunocomplexes may preferably be endotoxin and anti-endotoxin antibodies, thus reducing the number of different reagents necessary to carry out the method or be present in a kit for carrying out the method. Thus, an assay employing four tubes may be used to generate quantitative results, as described in copending application Ser. No. 09/457,465, incorporated herein by reference in its entirety. As will be described in more detail below, the assay tubes comprise a control and sample pair, and a maximal stimulatory amount of immunocomplexes, such as added (authentic) endotoxin and anti-endotoxin antibodies, and a corresponding control thereto.

In a further embodiment to the aforementioned 4-tube assay, as described in 09/353,189, filed Jul. 14, 1999, and incorporated herein by reference in its entirety, it was found surprisingly that the control for the maximum stimulatory amount of antigen could be omitted from the assay, and the resulting measurement providing a semi-quantitative readout which could readily discriminate between normal and elevated values of analyte such as a sepsis- or infection-related analyte, such as endotoxin. The details of this three-tube assay will be elaborated upon below.

The following discussion described particular embodiments of the invention set forth as a two-tube, four-tube, and three-tube method. While each format of the method (and corresponding kit) has particular features, many of the features are interchangeable, such as the nature of the bodily fluid, analyte, antibodies, stimulators, measurement method for oxidant production, phagocytic cells, etc., and the present invention and its various embodiments share and embrace all variations among these features. While each type of test is discussed, and examples described, with the scope of that test, it is understood that substitutions of various components may be made from other tests without deviating from the scope and intent of the invention. Moreover, the added assay for the maximal immunostimulatory level of immunocomplexes may be added to any other assay format to impart a more quantitative readout.

As noted above, non-limiting examples of bodily fluids useful in the practice of the invention include but are not limited to whole blood, plasma, serum, urine, saliva, and cerebrospinal fluid. Certain of these bodily fluids, such as whole blood, have been found to have adequate white blood cells normally present therein to provide the oxidant production proportional to the level of analyte in the sample, in combination with exogenously-added antibodies to the analyte. Adequate complement factors are also present in whole blood to provide an activator for the assay, although additional complement proteins as well as other activators may be added. Thus, whole blood may be used without supplementation of white blood cells, white blood cell fractions, white blood cells from cell culture, artificial or other oxidant-producing entities or components. Other samples which are normally free of or have low levels of white blood cells may be supplemented accordingly with oxidant-producing cells or components from another source. A preferred embodiment of the invention is the use of whole blood as the source of bodily fluid, chemiluminescence as the readout using a substrate such as luminol. A stimulator such as opsonized zymosan is also used to enhance oxidant production.

The invention is also directed to extracts of such cells as described above and to synthetic mixtures which contain the necessary components to generate a chemiluminescent response. Extract as used herein refers to any of those non-cellular systems described herein including both cellular extracts and such synthetic mixtures capable of generating oxidants in response to the presence and level of immunocomplexes, and may be used in combination with or as an alternative to white blood cells in and for any of the methods or kits described herein.

As noted above, the methods and kits of the invention have various uses in the health care field. In one aspect, a rapid test can be used in the emergency room or in the field (e.g., battlefield, field hospital, space station, remote field stations, etc.) to identify whether an individual is suffering from infection or perhaps more importantly, sepsis or sepsis syndrome. The results can guide treatment of a potentially and often rapidly fatal condition. Various parameters provided by the methods herein may help stage sepsis and indicate the best course of therapy based thereon. The test may also be used to monitor recovery and therapeutic interventions in the treatment of sepsis and infection. The measurement of sepsis-related analytes such as inflammatory cytokines is also useful in the monitoring of other diseases states in which elevated circulating or localized cytokines are indicative of disease, such as rheumatoid arthritis and Crohn's disease, as mere examples.

Notwithstanding the above, the foregoing description of various assay formats and kits are equally applied to analytes other than sepsis- and infection-related analytes in bodily fluids, as well as to measuring analytes in samples not derived from bodily fluids. As exemplified above, other analytes as readily measured in bodily fluids includes, hepatitis A, inflammatory mediators, drugs of abuse, therapeutic drugs, or cardiac markers, such as myoglobin, creatine kinase MB, troponin I or troponin T. Inflammatory mediators include but are not limited to tumor necrosis factor, interleukin-1, interleukin-6, interleukin-8, interferon, and transforming growth factor β.

The following descriptions of certain preferred embodiments of the invention are meant to be merely illustrative of non-limiting methods for carrying out the present invention.

### Two-tube Assay

The two-tube method has been described in U.S. Pat. 5,804,370, incorporated herein by reference in its entirety, and comprises:

i) incubating the test sample with an amount of test antibodies specific to a selected analyte to form antibody/marker complexes;

ii) allowing the antibody/marker complexes to interact with white blood cells or cell fractions or extracts which results in the production of oxidants; and

iii) measuring the amount of oxidant produced as an indicator of the presence or absence of the analyte in the sample.

For use in detecting a sepsis- or infection-related analyte, the assay comprises:

i) incubating the test sample with an amount of test antibodies specific to a selected sepsis- or infection-associated marker to form antibody/marker complexes;

ii) allowing the antibody/marker complexes to interact with white blood cells or cell fractions or extracts which results in the production of oxidants; and

iii) measuring the amount of oxidant produced as an indicator of the presence or absence of infection or sepsis.

In a particular embodiment, the measuring step is carried out using a chemiluminescent compound, which emits light proportional to the amount of oxidants in the sample. Oxidant-producing cells may be provided in the sample or added from an exogenous source. Complement factors similarly may be present in the sample or added. Optionally added activators may include zymosan, latex beads, opsonized zymosan or latex beads, a phorbol ester or FMPL. Thus, the assay of this embodiment further involves:

iv) introducing to the foregoing step(s) chemiluminescence compound to the test sample;

v) allowing the oxidants to react with the chemiluminescent compounds to emit luminescent light from the test sample;

vi) measuring the amount of emitted light over a predetermined period, and

vii) correlating the presence of the analyte (e.g., extent of infection) by comparison of the measured amount of emitted light of the test sample with measured amount of light emitted by a control sample which is treated the same as the test sample for steps i) to vi) except that in step i) control antibodies are used which are of the same class as the test antibodies but are non-specific to the analyte (e.g., sepsis or infection associated markers).

In accordance with another aspect of the invention, a diagnostic kit for use in determining the extent of infection or sepsis in a patient by detecting the presence of antigen indicative of infection or mediators in response to infection, in a patient's test sample comprises:

i) a first container of IgM, IgG or IgA antibody specific to analyte or mediators indicative of infection or sepsis;

ii) a second container of chemiluminescent compound; and

ii optionally, a third container of zymosan or latex beads, optionally opsonized; or a phorbol ester or FMLP.

An optional fourth container of oxidant-producing phagocytic cells may be included. Such cells may include lymphocytes, monocytes, immortalized leucocytes cells, or any combination thereof. An immortalized cell may be HL-60 cells. Oxidant-producing cell as used herein throughout also embraces other membrane-bounded vesicles or extracts or other artificial mixtures which contain or are prepared to contain the necessary machinery to generate oxidants in a similar fashion to white blood cells. Another contained may include the irrelevant but same-class antibody to be used as a control. The aforementioned kit for detecting sepsis or infection may also be used to detect any analyte by using antibodies specific to the analyte.

In order to provide a semi-quantitative estimate of the amount of endotoxin in the blood sample, the analysis is conducted, in accordance with one aspect of the invention, using 3 different dilutions of specific and control antibody each of which differ from the next highest concentration by one order of magnitude (i.e., 1:10, 1:100, 1:1000 dilution). The presence of antigen of interest, in this case, Gram-negative endotoxin, is confirmed by a statistically-significant increase in integrated light intensity or reaction slope during the first 10 to 20 minutes of reaction. The three different concentrations of antibody are used to discriminate and semi-quantitate the amount of endotoxin which is present. The principle of the triple concentration approach is based on the observation that maximal stimulation of chemiluminescence in whole blood occurs when antigen-antibody complementarily is optimal for the formation of macromolecular crosslinked immunocomplexes or aggregates. In the presence of high concentrations of antigen, a high antibody concentration is required to yield such optimal complementarily. Similarly, at intermediate and low concentrations of antigen less concentrated antibody is required for optimal complementarily and macromolecular aggregate formation. This basic principle has been used for years in Ouchterlony diffusion plates and radial diffusion plates for immunometric quantitation of precipitin reactions. The whole blood chemiluminescent approach provides a semi-quantitative determination of the antigen concentration in question as high, intermediate or low with analogous concentration range (i.e., ≧100 pg/ml, 20-100 pg/ml, ≦20 pg/ml). Thus, the maximal stimulation of chemiluminescence will occur for the 1:10 antibody dilution when the antigen level is at ≧100 pg/ml; for the 1:100 antibody dilution when the antigen level is at 20-100 pg/ml; and for the 1:1000 antibody dilution when the antigen level is at ≦20 pg/ml.

The examples below describe alternatives to these aspects, such as varying the order in which to add the reagents, varying blood dilutions, and omitting zymosan. However, the above aspects provide a better evaluation of the presence and degree of sepsis. Modifications of these protocols will still be within the scope of the invention. The whole blood sample may instead be a sub-fraction of white blood cells, such as neutrophils or lymphocytes or monocytes. A chemiluminescent compound other than luminol may be used, such as lucigenin or pholasin.

### Four-tube Assay

This aspect of the invention is a sensitive, specific and rapid general quantitative method for analytes present in blood. It has been described in U.S. Ser. No. 08/991,230, herein incorporated by reference in its entirety. As above, the method is based upon the specificity of antigen-antibody interactions and the high sensitivity of chemiluminescent light emission in response to oxidants produced from the interaction of immunocomplexes with white blood cell fractions in the presence of relevant complement proteins. The invention provides early, diagnostic, quantitative information for analytes such as those indicative of the extent of sepsis and the stage of sepsis. Results are obtained in minutes which is a great advantage over the previous time-consuming methods, for example, of blood culturing for determining the presence of sepsis-causing microorganisms.

To practice the method of the present invention, a sample from an individual is obtained, and divided into four aliquots. Two of the four aliquots are used to assess the chemiluminescent response of the white blood cells in the sample or those added thereto to immunocomplexes formed from the binding of any preselected analyte present in the sample with an antibody or antibodies to the preselected analyte which are added to the aliquot, the other aliquot used as a control. The second two aliquots are used to assess the overall response of the white blood cells present in, or added to, the sample to maximal stimulation by immunocomplexes, by adding a large amount of an antigen and its corresponding antibody to one of the aliquots, and only the antigen to the other aliquot as the control. The antigen used for maximal stimulation may or may not be the same as the analyte; for example, preferably, for convenience, endotoxin is used as the antigen, with anti-endotoxin antibodies, to stimulate the maximal response when the analyte being measured is endotoxin. An agent to generally enhance the chemiluminescent response optionally may be added to all of the aliquots, as well as a compound capable of producing light in response to the production of oxidants by white blood cells. Light emission from all four reaction aliquots is measured over a period of time. The amount of light produced by each aliquot is used to calculate the quantity of preselected analyte in the blood sample, based on a pre-established correlation between the amount of preselected analyte in the sample and the ratio between the integrated chemiluminescence of the four samples described above. Of course, pointed out above, other means for assessing oxidant production may be employed, though chemiluminescence is preferred.

It will thus be seen that the process of this aspect of the invention involves the following steps:

i) providing four aliquots of equal volume of a blood sample in which the level of a preselected analyte is to be determined;

ii) adding to one aliquot an amount of anti-analyte antibody sufficient to form an immunocomplex with said analyte in the sample;

iii) keeping one aliquot as a control to the aliquot described in step ii);

iv) adding to a third aliquot a maximum stimulatory amount of an antigen together with an amount of antibody sufficient to form a maximal amount of immunocomplexes with said antigen;

v) reacting a fourth aliquot with an amount of antigen equal to that added to the aliquot described in step iv);

vi) optionally adding to all four reaction aliquots an agent to enhance oxidant production, such as opsonized zymosan or latex particles;

vii) incubating the four reaction aliquots for a time sufficient for any immunocomplexes formed in the samples to react with the white blood cells and complement proteins in the plasma to produce oxidants;

viii) contacting a chemiluminescent compound which reacts with the oxidants to generate light with all four reaction aliquots, prior to or after step vi);

ix) measuring light emission from the four reaction aliquots over a predetermined time period; and

x) correlating differences in light emission among the four reaction aliquots to determine the quantity of the preselected analyte in the sample.

The various components of the assay are those described above in the corresponding two-tube assay, including the optional activator and exogenous source of cells.

In accordance with another aspect of the invention, a diagnostic kit is provided for quantitating a preselected analyte in a patient's blood sample. In one embodiment, the kit may be used to determine the extent of infection in a patient by quantitating an analyte indicative of infection or mediators in response to infection, in a patient's blood sample containing white blood cell fractions comprising:

i) a first container of IgM, IgG or IgA antibody specific to an analyte or mediators indicative of infection;

ii) a second container of chemiluminescent compound;

iii) a third container of antigen; and

iv) a fourth contained of anti-antigen antibodies.

An agent to enhance the chemiluminescent response, such as zymosan or opsonized zymosan, latex or opsonized latex, phorbol ester, FMLP, or complement factors may be included in another container in the kit. A source of exogenous oxidant-producing phagocytic cells or a cell extract may also be included in the kit.

### Three-tube Assay

This aspect of the invention has been described in U.S. Ser. No. 09/353,189, incorporated herein by reference in its entirety. It is directed to a method for measuring the level of a preselected analyte present in a sample of a bodily fluid comprising the following steps

i) providing three aliquots of the sample, designated aliquots A, B, and C;

ii) providing a source of oxidant-producing phagocytic cells or extract thereof and a source of complement proteins;

iii) providing aliquot B with an amount of anti-analyte antibody sufficient to form an immunocomplex with the analyte in the sample, to provide reaction aliquot B;

iv) providing aliquot A as a control to reaction aliquot B without added anti-analyte antibody, to provide reaction aliquot A;

v) providing aliquot C with a equivalent amount of the anti-analyte antibody as in reaction aliquot B, and in addition containing a maximal stimulatory amount of an analyte, to provide reaction aliquot C;

vi) incubating reaction aliquots A, B, and C with oxidant-producing phagocytic cells and a source of complement proteins under suitable conditions and for a time sufficient for any immunocomplexes formed in the reaction aliquots to react with oxidant-producing phagocytic cells and complement proteins to produce oxidants;

vii) contacting a chemiluminescent compound which reacts with the oxidants to generate light with reaction aliquots A, B, and C, prior to or after step vi);

viii) measuring light emission from reaction aliquots A, B, and C over a predetermined time period under suitable conditions; and

ix) correlating differences in light emission among reaction aliquots A, B, and C as an indicator of the amount of analyte in the sample.

Of course, as described hereinabove, the foregoing embodiment utilizing chemiluminescence as a measure of oxidant production is a preferred embodiment though others are embraced within the scope of the present invention.

The aforementioned steps may be carried out following manual, semi-automated, or automated procedures. The test may provide results in a short period of time, such that the measurement of the analyte can be performed to aid in the rapid diagnosis of a patient's condition. Instrumentation may be provided that can be performed in the emergency room, at the bedside, or for home use. Depending on the assay format, a test may be performed in around 20 minutes or less. The various components of the method are those as described hereinabove.

In a further aspect of the present invention, a diagnostic kit for measuring the level of a preselected analyte present within a sample of a bodily fluid is provided, comprising:

(i) a first container of IgM, IgG or IgA antibody specific to the preselected analyte;

(ii) a second container of chemiluminescent compound; and

(iii) a third container of analyte.

A source of oxidant-producing phagocytic cells or cell extract may be included in the kit for samples which do not contain them; the cells may be neutrophils, lymphocytes, monocytes, immortalized cells, or combinations thereof. The diagnostic kit may also include additional container containing an agent capable of increasing oxidant production by white blood cells on exposure to immunocomplexes, for example, zymosan, latex particles, phorbol ester, N-formyl-mel-leu-phe, opsonized zymosan, opsonized latex particles, or combinations. Complement factors may also be included. The chemiluminescent compound may be luminol, lucigenin or pholasin.

The invention herein in its various forms is also broadly is directed to a method for determining the stage of sepsis of a patient from a sample of whole blood comprising the concurrent measurement of: (a) the level of microbial products or inflammatory mediators; (b) the maximum oxidant production by the patient's neutrophils; and (c) the level of responsiveness of the patient's neutrophils to a maximum stimulatory level of immunocomplexes. These parameters are measured as described in the previous methods and the citations therein.

The reagents of the methods and kits described herein may be provided in the form of lyophilized reagent beads, such as described in copending application Ser. No. 09/353,191, incorporated herein by reference in its entirety.

The following examples are illustrative but non-limiting descriptions of various ways in which the invention herein may be carried out.

### EXAMPLE 1

### Optimization of Chemiluminescent Response of Two Different Endotoxin Concentrations (100 pg/ml and 1000 pg/ml) by Varying the Antibody Concentration

Three 1 ml samples of whole blood anticoagulated with EDTA collected from one donor were mixed with 10 μl of HBSS. One of the 10 μl aliquots of HBSS contained 100 pg of endotoxin and the other 10 μl aliquot contained 1000 pg of endotoxin. Each sample either with or without endotoxin was then diluted tenfold with HBSS containing 2 U/L of sodium heparin. The following assay protocol was then used: 200 μl of luminol solution, 100 μl of 10X diluted blood, 25 μl of monoclonal antibody against endotoxin and 50 μl of complement opsonized zymosan. The final concentration of monoclonal antibody in the reaction mixture was varied from 0.2 μg/ml to 0.0025 μg/ml in dilution increments of 3 fold. All assays were analyzed in triplicate and the reactions were initiated by the addition of opsonized zymosan to the reaction mixture. The chemiluminescent response was monitored for 50 minutes at 37° C. Chemiluminescent curve integrals were taken from the time of zymosan addition until 5 minutes of the initial acceleration phase of the reaction for comparison of responses. All integrals were compared to the parallel control containing an equivalent concentration of monoclonal antibody but no endotoxin.

The possibility that one antibody concentration could span a range of CL response from 0 to 1000 pg/ml of endotoxin was investigated. After careful inspection of the data obtained over a 50 minute assay period it was observed that the best signal to noise ratio was achieved by considering CL curve integrals over the first five minute acceleration phase of the reaction. This data is tabulated in the “Integral” column of Table 4. The starting antibody dilution in this experiment was 0.2 μg/ml. All subsequent dilutions of antibody were made in threefold steps. It is clear from this data that the maximal response ratio between control cuvettes with no endotoxin and cuvettes containing blood with an endotoxin concentration of 100 pg/ml was achieved at the highest concentration of antibody tested, namely 0.2 μg/ml. The response ratio at this concentration was 2.1. At an LPS concentration of 1000 pg/ml, the maximal response ratio was achieved at an antibody concentration, of 0.007 μg/ml. At this antibody concentration, the response ratio for the 1000 pg/ml standard was 1.7.

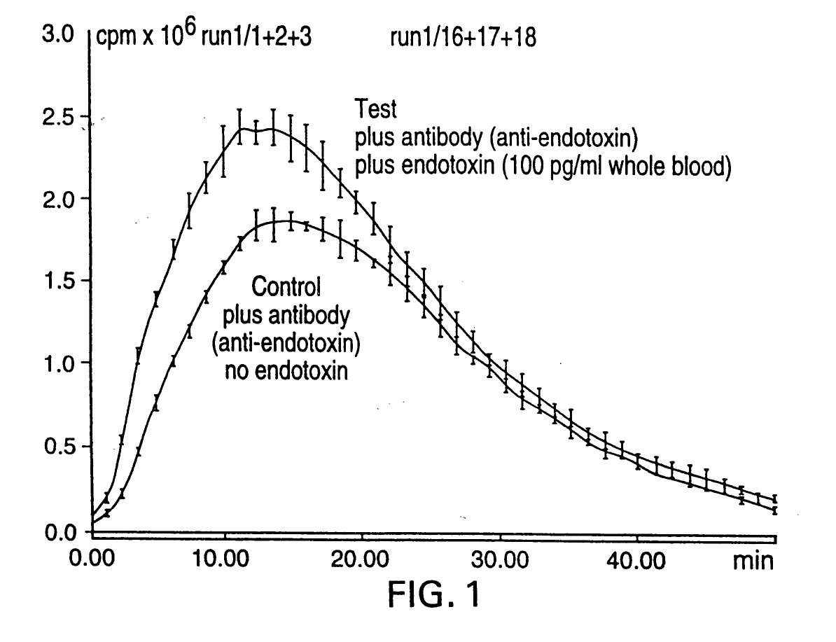

FIG. 1 graphically presents the CL data obtained from the reaction mixtures which gave the largest response ratio for endotoxin at a concentration of 100 pg/ml of whole blood. The plotted data emphasizes the difference in the initial slope of the reactions and in the CL maxima. An adequate differentiation of the signals was clearly evidenced after only 5 minutes of reaction emphasizing the rapid diagnostic potential of the assay. The standard chemiluminometer measures emitted luminescent light by use of the standard type of electronic photo counter. Periodically as plotted along the X-axis in minutes, the light emission is measured based upon the photon counts per minute (cpm). The cpm value is then plotted on the Y-axis whereby over time the receptive curves are developed. In summary, the presence of endotoxin in the sample results in a steeper reaction slope during the acceleration phase of the reaction and a higher CL maximum light emission. In many samples, the time to reach CL maximum is shortened by the presence of endotoxin.

### Example 2

### Initial Correlation Analysis Between Chemiluminescent Assay of Endotoxin and a Standard Reference Method Employing the Limulus Amebocyte Lysate (LAL) Assay

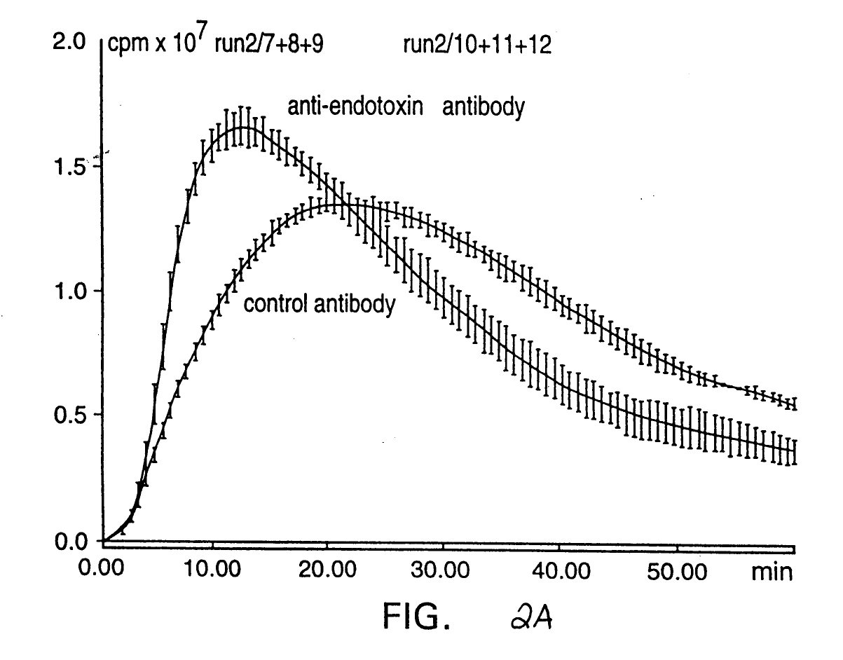

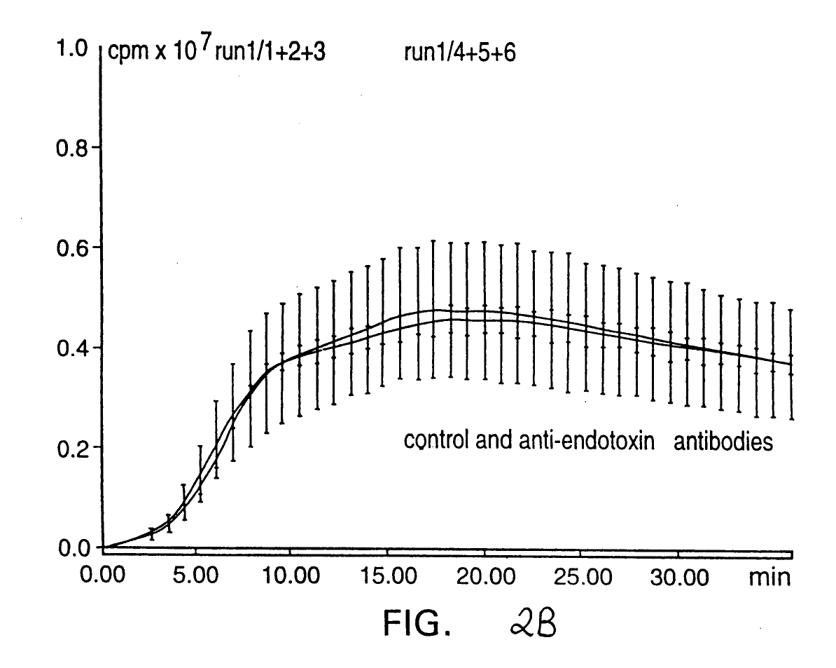

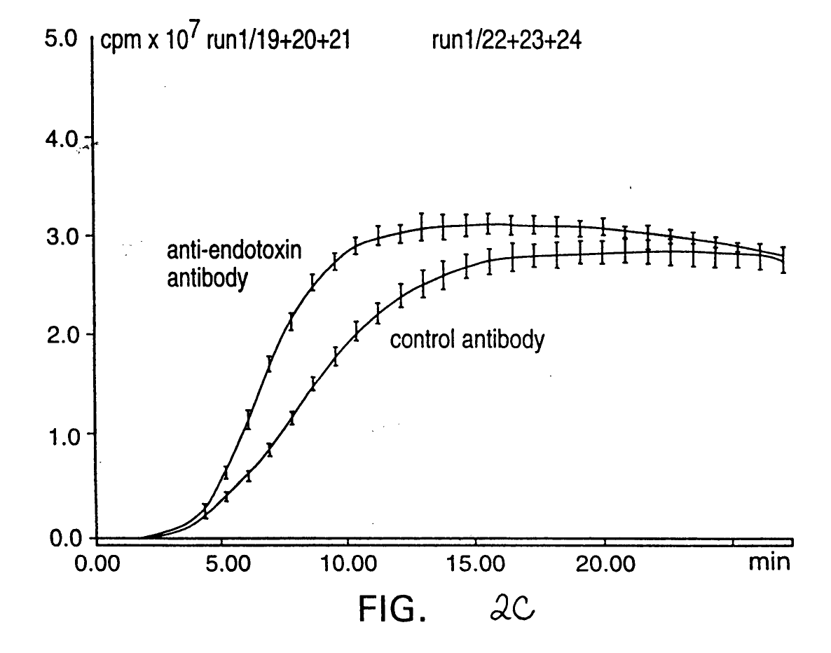

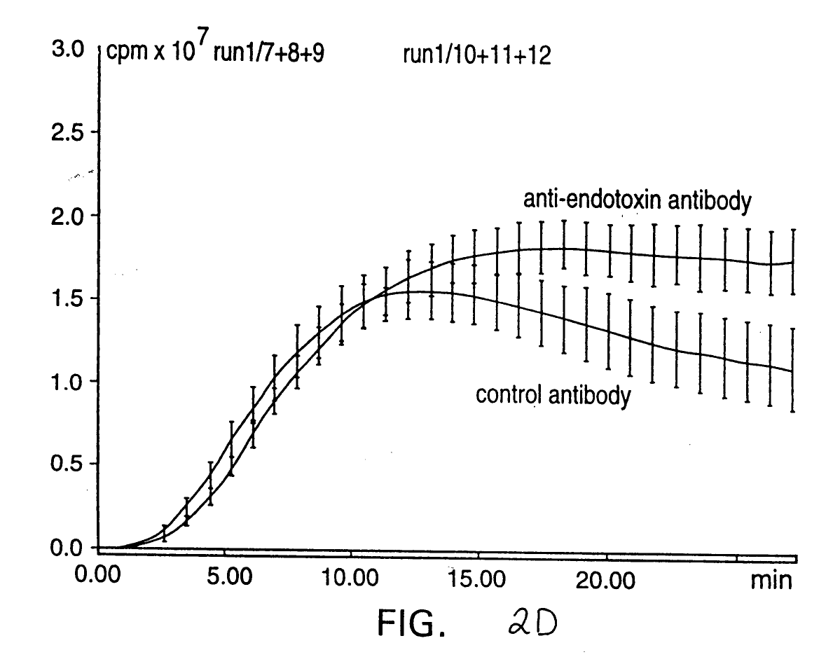

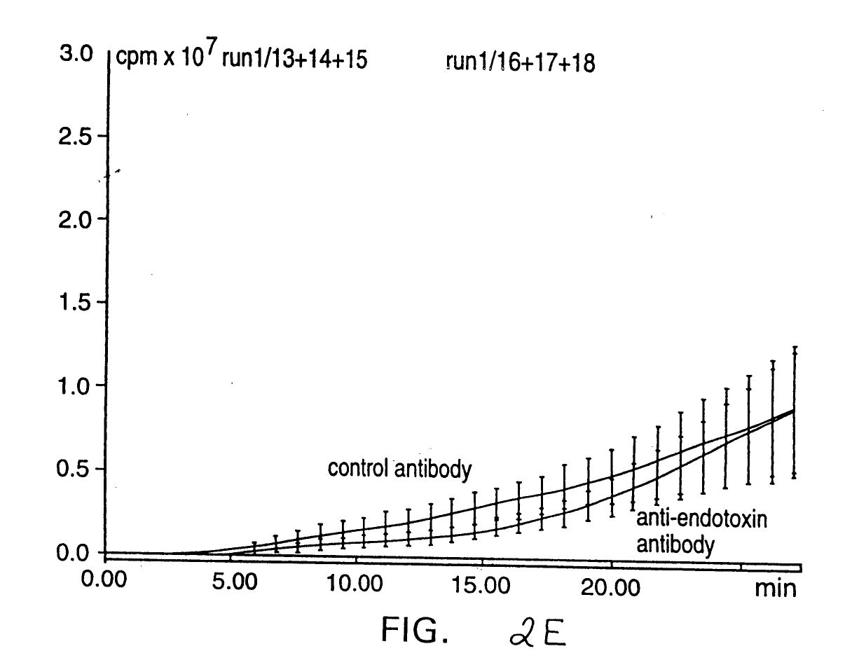

In this study, arterial blood samples were taken from patients with clinical symptoms of sepsis into sterile EDTA-containing Vacutainer tubes and assayed for the presence of endotoxin by both the chemiluminescent whole blood assay and the reference Limulus amebocyte lysate assay using assay kits purchased from BioWhittaker (Walkerville, Md., U.S.A.) or Seikagaku Kogyo Ltd. (Tokyo, Japan). Control samples were also obtained from non-septic patients and healthy ambulatory donors. FIG. 2A displays the chemiluminescent response of blood taken from the radial artery of a patient with severe sepsis syndrome who had died 6 hours after the sample was taken. The cause of death was hypotensive shock which was refractory to inotropic support. It is clear from the CL response in the presence of anti-LPS antibodies that this patient had a high level of endotoxin which was confirmed by LAL assay to be on the order of greater than 700 pg/ml (see Table 8). Even with such high levels of antigen which would result in high levels of mediators and thereby white blood cell activation, the antigen/antibody formation still causes an increase in white blood cells oxidant production. FIG. 2B illustrates the CL profile of a healthy ambulatory volunteer and shows no differential response to anti-LPS antibody which was confirmed by LAL assay to indicate the absence of LPS in the blood. FIG. 2C displays the CL response of a patient with chronic sepsis which was confirmed by blood culture to be primarily due to a beta hemolytic gram positive streptococcus. The CL assay indicated that this patient also had a response consistent with a low level of Gram-negative septicemia which was below the limits of detection when assayed by LAL. The limit of detection using the Seikagaku Kogyo Endospecy LAL assay was a whole blood concentration of 50 pg/ml LPS. In order to remove interfering substances this LAL assay requires a perchloric acid pre-treatment step which results in a tenfold dilution of the blood which is added to the assay mixture. This step poses a major limit on the analytical sensitivity of the assay. FIG. 2D displays the CL response of a patient who had severe sepsis syndrome which ultimately contributed to his death 3 days after the blood sample used for the analysis was taken. The CL analysis indicated no evidence of LPS in the blood which was confirmed by LAL assay. The microbiological reports on culture material for this patient suggested that he had gram positive sepsis. FIG. 2E represents the results of CL assay for LPS conducted on blood obtained from a patient who was being weaned from respiratory support and was previously cachectic, but had no clinical evidence of any septic foci. The LAL assay confirmed the absence of endotoxin. These results suggest that the CL assay devised for the rapid detection of Gram-negative endotoxin is capable of detecting LPS in patents with sepsis syndrome in whom LPS is detectable by standard LAL assay. In one patient (FIG. 2C), Gram-negative endotoxin was detectable by CL assay but probably below the limits of detection based on the LAL assay. The sensitivity and rapidity of the CL assay confirms its great potential in the early detection and clinical management of patients with sepsis syndrome.

The chemiluminescence assay mixture was composed of 50 μl of undiluted anti-coagulated whole blood, 50 μl of antibody (concentration 0.2 mg IgM/ml) and 200 μl of luminol solution and 50 μl of complement opsonized zymosan. All reagents were added in the order listed and the first two solutions were pre-incubated at 37 ° C. for 5 minutes prior to the addition of luminol and zymosan, followed by the initiation of CL readings which were monitored for up to 60 minutes. All chemiluminescence assays were always run in conjunction with blood obtained from non-septic patients and ambulatory lab staff to verify the absence of false positive results. A positive control sample containing blood supplemented in vitro E. coli LPS at a concentration of 100 pg/ml was always assayed with each run of patient samples.

Parallel blood samples from patients and controls were centrifuged at 700×g for 15 minutes to remove cells and duplicate 50 μl aliquots of plasma were removed using endotoxin free pipettes and transferred into endotoxin-free glass test tubes for LAL assay. The plasma was treated with endotoxin free perchloric acid to remove inhibitory factors according to the procedure of Inada K., et al. CRC Review on Gram-negative Endotoxin 225 (1989) and subsequently assayed for endotoxin using the high sensitivity protocol as specified by Seikagaku Kogyo Inc (Toxicolor System Instruction Manual for Endotoxin Determination). The endotoxin levels were also confirmed using the LAL assay protocol for human plasma as specified by BioWhittaker.

In a further comparison of the present invention's CL method and the LAL assay, patients were tested for the presence of LPS at different times and using varying antibody dilutions. The LPS values for the CL assay for each test closely matched the values for the LAL assay. These LPS results for the CL assay and LAL assay are shown in Table 1. These samples were assayed using both LAL assays (Seikagaku and BioWhittaker). The BioWhittaker assay was found to be sensitive below 50 pg/ml of LPS as compared to the Seikagaku assay protocol.

TABLE 1
COMPARISON OF LPS RESULTS BETWEEN CL METHOD
AND LAL ASSAY IN PATIENTS WITH CLINICAL SEPSIS

| | CL Assay Result | | LAL Assay |
|---|---|---|---|
| Patient | pg/ml LPS | Ab Dilution | pg/ml |
| M. O. | >100 | 1:10 | 130 |
| M. O. | 20-50 | 1:100 | 40 |
| M. O. | >200 | 1:10 | 400 |
| J. S. | 20-50 | 1:100 | 50 |
| J. S. | Neg. | | Neg. |
| J. S. | >100 | 1:10 | 90 |
| M. P. | >100 | 1:10 | 120 |
| P. S. | Neg. | | Neg. |
| P. S. | Neg. | | Neg. |
| P. S. | Neg. | | Neg. |
| J. V. | >200 | 1:10 | >700 |
| J. V. | >200 | 1:10 | 750 |
| M. H. | 20-50 | 1:100 | 60 |

### Example 3

### Chemiluminescent Response of Whole Blood from a Septic Patient Using Three Concentrations of Antibody

The patient had recurrent problems with a leaky duodenal ulcer. The patient experienced a temperature spike in the morning. The blood sample was taken approximately four hours before he was taken to the OR for abdominal cavity lavage.

A preferred approach for testing patient samples for endotoxin is based upon the following assay conditions: 20 microliters of the patient's blood (EDTA anti-coagulated) is mixed with 20 μl (microliter) of antibody (three different dilutions are used, 0.2, 0.002 and 0.002 mg/ml) in an endotoxin free assay cuvette. The mixture is incubated for 10 minutes at 37° C. and then 200 μl of luminol solution (40 μM) is added (pre-equilibrated to a temperature of 37°0 C.) followed by 50 μl of complement opsonized zymosan 2.5-3.0 ×10⁹ particles/ml. Measurement of emitted light is then initiated in the chemiluminometer.

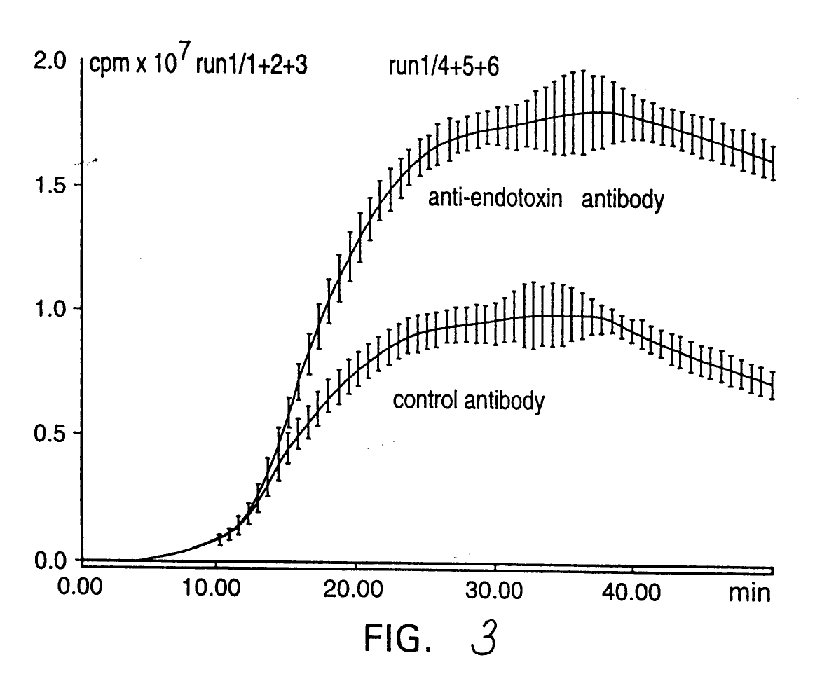

As demonstrated in FIG. 3 (using the preferred patient assay format) a significant difference between control and anti-endotoxin antibodies can be achieved within 20 minutes. The assay is shown only for the antibody concentration of 0.2 mg/ml since the other antibody concentrations gave no differential response between control and anti-endotoxin antibody. The upper tracing in the Figure depicts the CL response of anti-endotoxin antibody containing blood, while the lower panel depicts the pattern achieved with a non-specific control antibody. The patient's sample was confirmed to contain 420 pg/ml of Gram-negative endotoxin in LAL assay. The format of this assay was designed to minimize the amount of antibody necessary to evoke a significant chemiluminescence enhancement in the presence of Gram-negative endotoxin. For this reason only patient sample and the antibody are incubated in the first phase of the reaction sequence in order to maximize effective antibody antigen complex formation. This preferred format has been adopted for patient studies.

FIG. 3 demonstrates clearly the difference in the chemiluminescence levels of the patient as compared to the control using an antibody concentration of 0.2 mg/ml.

### Example 4

### Quantitative use of the Whole Blood Chemiluminescence Assay in the Detection of Gram Negative Endotoxin (LPS).

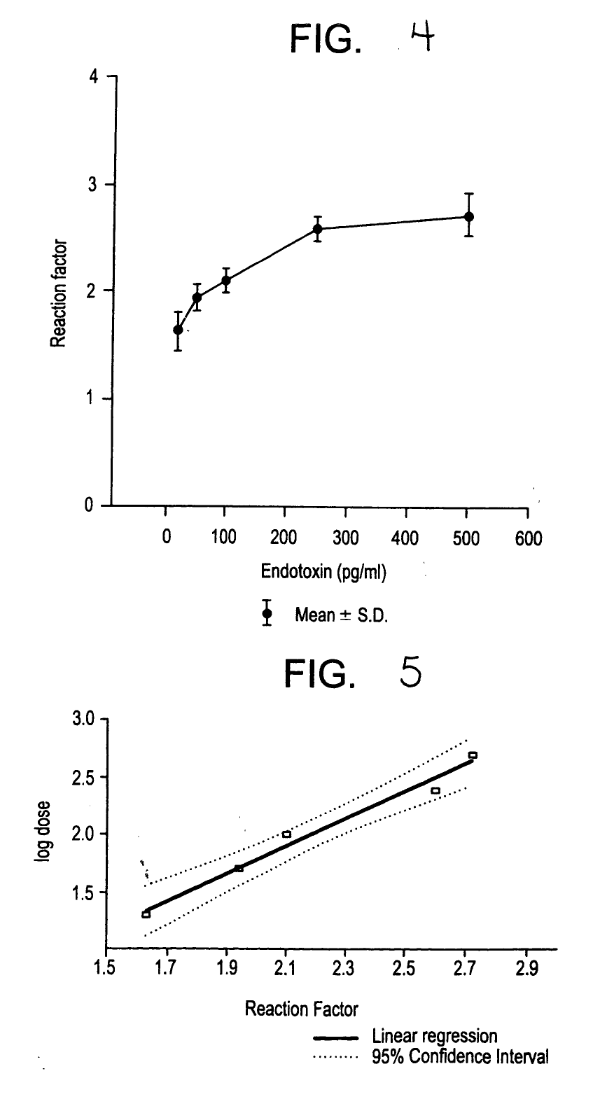

The ability of the Xomen-E5 antibody to yield a quantitative assay of endotoxin in whole blood at a fixed concentration of antibody was investigated. In this assay strategy, an assay mixture was employed containing 50 μl of antibody (either Xomen-E5 or non-specific control both at a concentration of 0.05 mg/ml) which was mixed with 16 μl of whole blood and incubated at room temperature for 5 minutes. To this mixture was added a luminol-containing buffer solution (600 μl) which was warmed to 37° C. and 50 μl of human complement opsonized zymosan. All samples were assayed in triplicate with control and Xomen-E5 antibody. To three separate blood samples obtained from three endotoxin free donors (including on ICU patient and two lab volunteers) varying concentrations of E. coli endotoxin were added yielding final endotoxin concentrations of 20, 50, 100, 250 and 500 pg/ml of whole blood. These blood samples were assayed utilizing the protocol above with control and anti-endotoxin antibodies. Total light integrals were obtained for the mean reaction curves for the anti-endotoxin and control antibody containing samples at 20 minutes of total reaction time. For each endotoxin concentration the light integral for the control antibody-containing samples was subtracted from the light integral of the E5 antibody containing samples and divided by the light integral of the control antibody-containing samples to normalize for differences in white cell count and white cell reactivity. This calculation yielded a “reaction factor” which was then plotted against the endotoxin concentration. The relationship between the reaction factor and antibody concentration is displayed in both linear and semi-logarithmic form. It is therefore possible to use the reaction factor calculated from patient samples to interpolate the calibration curve and hence estimate the endotoxin concentration contained within an unknown sample. Results are shown in FIGS. 4 and 5.

### Example 5

### Four-tube Assay: Quantitation of LPS

Reagents and bacterial products. Luminol (5-amino-2,3-dihydro-1,4-phthalazinedione, free acid), zymosan A (Saccharomyces cerevisiae), lipopolysaccharides from Escherichia coli (E. coli) serotypes (026:B6, 055:B5, 0111:B4) (Gram-negative endotoxin), and lipoteichoic acids from Streptococcus spp. (Gram-positive cell wall constituent) were purchased from Sigma (Sigma Chemical Co., St. Louis, Mo.).

Chemiluminescence Reagents. Buffer for measurement of whole blood or white cell chemiluminescence studies was HBSS (pyrogen free, endotoxin less than 0.005 EU/ml) containing 1.5 mM calcium salt and 0.9 mM magnesium salt (Gibco BRL, Grand Island, N. Y.). This buffer (500 ml) was vigorously mixed overnight at 25° C. with luminol to yield a saturated solution (150 μM, HBSS-luminol) and then supplemented with 4 U/ml of lithium heparin.

Opsonized Zymosan. To prepare human complement-opsonized zymosan, pooled fresh frozen citrate anti-coagulated human plasma was dialyzed against 4 volumes of 28.5% saturated ammonium sulfate solution for 2 hours at room temperature and then against fresh 28.5% saturated ammonium sulfate overnight at 4° C. The precipitate was removed by centrifugation and the supernatant dialyzed against 2 changes of 10 volumes of HBSS without calcium and magnesium at 40° C. This immunoglobulin-depleted serum fraction (<10% IgG and IgM based on nephelometric assay) was then mixed with a half volume of heat-activated zymosan A (5 g/liter of normal saline) in the presence of 1.3 mM calcium salt and 0.9 mM magnesium salt for 15 minutes at room temperature to opsonize the zymosan. The opsonized zymosan was subsequently washed three times with 2 volumes of ice-cold sterile normal saline and resuspended in its original volume (approx. 3×10⁶ particles per microliter).

Chemiluminescent Assay for Endotoxin. All glass surfaces used for endotoxin assay or storage of reagents for endotoxin assay including assay tubes were depyrogenated by heating to 300° C. for at least 6 hours. All polystyrene and polyethylene surfaces used for storage of antibodies, HBSS-luminol or blood products were sterile and essentially endotoxin free as determined by chromogenic LAL assay of pyrogen free water left in contact with the surface of interest. All pipette tips used for fluid transfer were sterile and pyrogen free (Diamed, Mississauga, Ontario, Canada). Blood samples used for the assay were drawn by venipuncture or through indwelling arterial lines into sterile 3 ml EDTA anti-coagulated Vacutainer tubes (Becton Dickenson, Franklin Lakes, N.J.) which were pretested for LPS content (less than 0.005 EU/ml).

All chemiluminescence experiments utilizing whole blood or blood cell fractions were assayed in triplicate and the results expressed as the mean luminometer counts per minute ±1 SD. In all assays, HBSS-luminol buffer (300 μl) was pre-mixed with 30 μl of antibody solution and subsequently incubated with 10 μl of whole blood or isolated neutrophils in fresh human plasma. After incubation with blood at 37° C. for 5 minutes in a thermostatted aluminum heating block the assay tubes were transferred to the chemiluminometer (E. G. & G. Berthold Autolumat LB953, Wildbad, Germany) for addition of 20 μl of human complement-opsonized zymosan. All assays were incubated at 37° C. in the chemiluminometer for 20 minutes with continuous measurement of light emission from each tube at least every 60 seconds for a minimum 0.6 second counting window. Chemiluminescence reaction curves and integrals were captured using Axis Cellular Luminescence System Software (version 1.03 from ExOxEmis Inc., San Antonio, Tex.).

To permit quantitation of endotoxin in whole blood, the following reaction aliquots were set up:

A = Whole blood + zymosan
B = Whole blood + anti-LPS antibody + zymosan
C = Whole blood + exogenous LPS (800 pg/ml) + zymosan
D = Whole blood + exogenous LPS (800 pg/ml) + zymosan +
anti-LPS antibody.

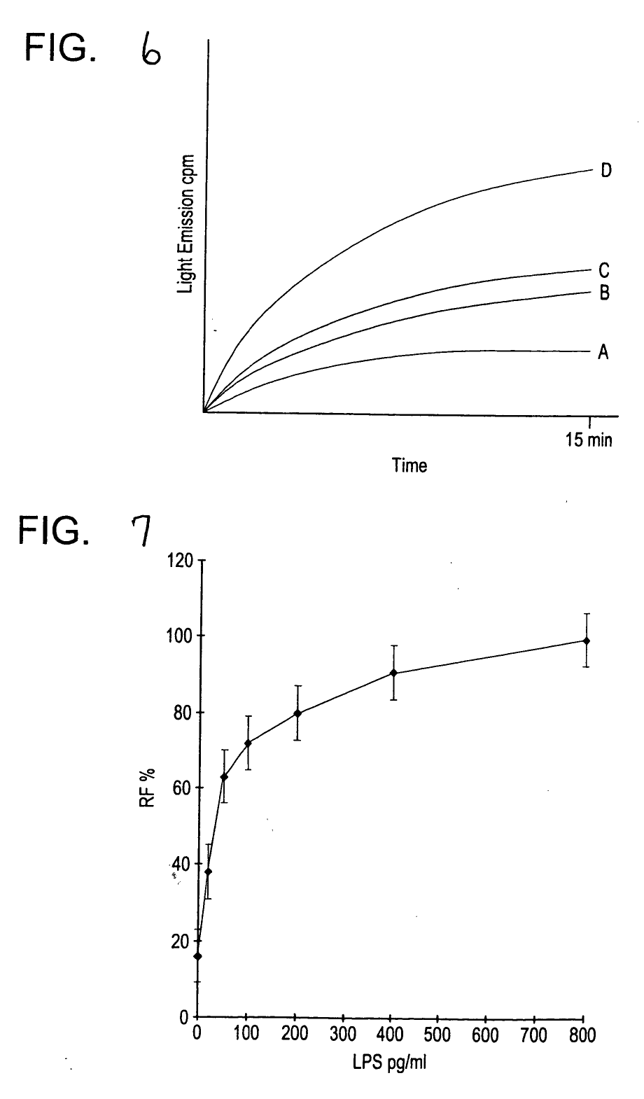

All reaction aliquots contained opsonized zymosan in order to optimize oxidant production of the patient's white blood cells in response to immunocomplexes. In addition to the patient's blood sample and zymosan, tube B contained antibody against the analyte to be measured, in this case endotoxin. Tube A served as a control to tube B. In order to determine the maximal response of the patient's white blood cells to immunocomplexes, tube C contained the maximal stimulatory concentration of LPS from E. coli 055:B5 plus anti-endotoxin antibody (determined to be 800 pg/ml or 0.67 EU/ml at an antibody concentration of 0.8 μg/assay); control tube D contained the same amount of antigen but no antibody. While in this example the antigen used to form immunocomplexes to determine maximal response (endotoxin-anti-endotoxin) was identical to the analyte, this does not need to be the true for all analytes. The response factor, RF=∫(B-A)/∫(D-C), was calculated as the difference between the antibody-dependent (tube B) and non-antibody-dependent (tube A) twenty-minute reaction integrals divided by the difference in antibody-dependent (tube D) and non-antibody-dependent (tube C) twenty-minute reaction integrals of reaction mixtures containing a maximal stimulatory dose of endotoxin. A typical whole blood chemiluminescence profile of a patient with endotoxemia is shown in FIG. 6.

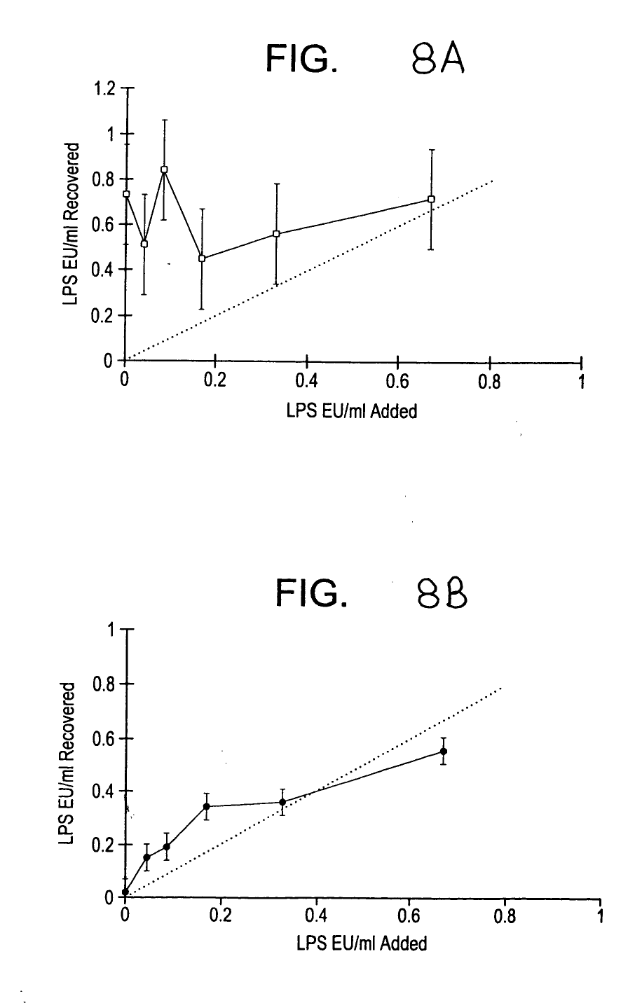

The averaged standard % RF curve established with 40 non-endotoxemic blood samples is displayed in FIG. 7. At the antibody concentration employed in the assays depicted in FIG. 2 (0.8 μg protein), a sharp dose-response curve was achieved between 0 and 80 pg/ml, then a more gradual response was seen over a range of 80 to 400 pg/ml with a plateau being achieved at 800 to 2000 pg/ml.

### Example 6

### Clinical Application of the Assay for Endotoxin Measurement

To validate the utility of whole blood chemiluminescence for quantitating endotoxin levels in patient's blood, evaluating white blood cell immunoresponsiveness, and determining the association between endotoxemia and clinically-important outcomes for critically ill patients, whole blood endotoxin measurements by the method of the present invention were made on 74 consecutive patients upon admission to a medical surgical intensive care unit. A total of 101 patients who met sepsis criteria as defined by ACCP/SCCM consensus were prospectively studied. Daily assays in triplicate were obtained.

Characteristics of Patients by Intensive Care Unit Admission Diagnosis

| | | | | |
|---|---|---|---|---|
| | | Number of | | |
| | Number of | patients with | | |
| | patients with | Endotoxin | | |
| Diagnosis | diagnosis | >50 pg/ml | Prevalence | Mortality |

Sepsis patients:

| | | | | |
|---|---|---|---|---|
| Sepsis | 95 | 64 | 67% | 52% |

Non-sepsis patients:

| | | | | |
|---|---|---|---|---|
| Elective | 21 | 9 | 45% | 0% |
| Surgery | | | | |
| Single Organ | 14 | 4 | 29% | 29% |
| Failure | | | | |
| Post Arrest | 6 | 4 | 67% | 67% |
| Other | 8 | 3 | 33% | 33% |

Control patients (n=30) had no detectable endotoxin. Patents categorized in the non- sepsis group had mean endotoxin levels of 226±345 pg/ml in the blood. Patents categorized in the sepsis group had mean levels of 404±354 pg/ml (p=0.05 vs. the non-sepsis group).

The following conclusions may be drawn from these data: (1) Endotoxemia is associated with conditions other than sepsis. A significant number of patients not diagnosed with sepsis had levels of endotoxin above 50 pg/ml (for example, 9 of 21 or 45% of patients for elective surgery; 4 of 6 or 67% of post-arrest patients). Patients with sepsis had almost a two-fold average increase in endotoxin levels. Also, patients with elevated endotoxin levels (>50 pg/ml) had a higher risk of mortality (p<0.05).

Early, accurate detection of endotoxemia may allow prompt intervention with anti-sepsis, or anti-endotoxin strategies and could result in altering the progression of the inflammatory response through sepsis to organ dysfunction and shock.

### Example 7

### Measurement of LPS Using The Three-tube Assay

To measure the amount of endotoxin in a sample of whole blood, the following reaction aliquots were prepared:

A = Whole blood + zymosan
B = Whole blood + zymosan + anti-LPS antibody
C = Whole blood + zymosan + anti-LPS antibody +
exogenous LPS (800 pg/ml)

All reaction aliquots contained zymosan in order to optimize oxidant production of the patient's white blood cells in response to immunocomplexes. In addition to the patient's blood sample and zymosan, tube B contained antibody against the analyte to be measured, in this case endotoxin. Tube A served as a control to tube B. In order to determine the maximal response of the patient's white blood cells to immunocomplexes, tube C contained a maximal stimulatory amount of immunocomplexes, derived from the same amount of anti-endotoxin antibody as in tube B, with the addition of LPS from E. coli 055:B5 (determined to be 800 pg/ml or 0.67 EU/ml at an antibody concentration of 0.4 μg/assay). While in this example the antigen used to form immunocomplexes to determine maximal response (endotoxin-anti-endotoxin) was identical to the analyte, this does not need to be the true for all analytes, although it is most convenient to do so.

The following materials were used and methods followed in carrying out the assay. Variations in the components described here as well as the procedures may be modified by standard procedures without deviating from the invention.

All glass surfaces used for endotoxin assay or storage of reagents for endotoxin assay including assay tubes were depyrogenated by heating to 300° C. for at least 6 hours. All polystyrene and polyethylene surfaces used for storage of antibodies, HBSS-luminol or blood products were sterile and essentially endotoxin free as determined by chromogenic LAL assay of pyrogen free water left in contact with the surface of interest. All pipette tips used for fluid transfer were sterile and pyrogen free (Diamed, Mississauga, Ontario, Canada). Blood samples used for the assay were drawn by venipuncture or through indwelling arterial lines into sterile 3 ml EDTA anti-coagulated Vacutainer tubes (Becton Dickenson, Franklin Lakes, N.J.) which were pretested for LPS content (less than 0.005 EU/ml).

Luminol (5-amino-2,3-dihydro-1,4-phthalazinedione, free acid), zymosan A (Saccharomyces cerevisiae), lipopolysaccharides from Escherichia coli (E. coli) serotypes (026:B6, 055:B5, 0111:B4) (Gram-negative endotoxin), and lipoteichoic acids from Streptococcus spp. (Gram-positive cell wall constituent) were purchased from Sigma (Sigma Chemical Co., St. Louis, Mo.).

Buffer for measurement of whole blood or white cell chemiluminescence studies was HBSS (pyrogen free, endotoxin less than 0.005 EU/ml) containing 1.5 mM calcium salt and 0.9 mM magnesium salt (Gibco BRL, Grand Island, N.Y.). This buffer (500 ml) was vigorously mixed overnight at 25° C. with luminol to yield a saturated solution (150 μM, HBSS-luminol) and then supplemented with 4 U/ml of lithium heparin.

All chemiluminescence experiments were assayed in triplicate and the results expressed as the mean luminometer counts per minute ±1 SD. Assays may also be prepared using duplicate or single tubes for reaction tubes A, B and C.

The following assay protocol was followed. Two aliquots of blood (500 μl) are dispensed into depyrogenated glass tubes into a thermostatted aluminum block pre-heated to 37° C. One tube contained a maximal dose of LPS; the other tube is empty. These tubes are incubated for 10 min. at 37° C. During the last 5 minutes of this incubation glass or polystyrene assay tubes are loaded into the heating block. Three tubes are used per assay. Tube A contains control reagent used for antibody stabilization or no reagent at all, Tubes B and C contain antibody. To each tube a mixture of Luminol Buffer with unopsonized zymosan is added (500 μl per tube). This mixture is temperature equilibrated for at least 5 min. After the blood has incubated for a total of 10 min. at 37° C., 20 μl is transferred into assay tubes A and B from the blood tube with no LPS and 20 μl is transferred from the blood tube containing LPS into assay tube C. All tubes are vortexed and placed in the chemiluminometer for reading. The luminometer is thermostatted at 37° C. and the assay is read for a total of 20 min.

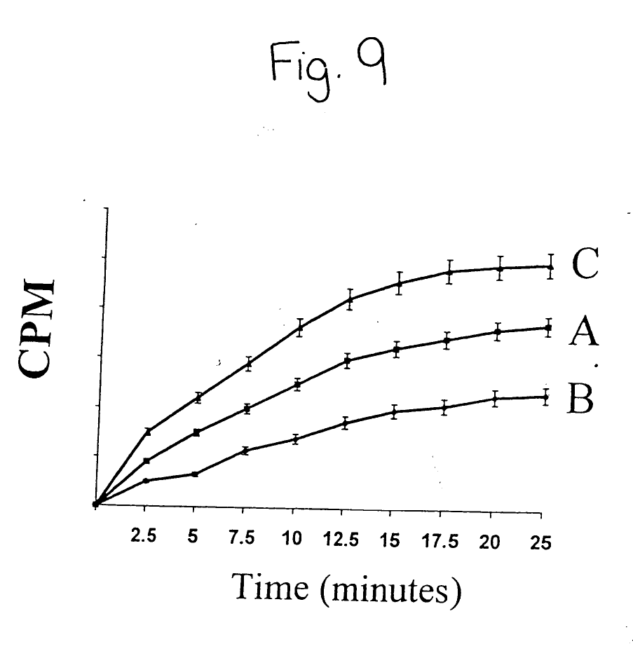

A typical whole blood chemiluminescence profile of a patient with endotoxemia is shown in FIG. 8. The 20-minute light integrals of tubes A, B and C are used to calculate the amount of LPS in the sample as follows. The amount of LPS present in the sample is referred to as “Endotoxin Activity” (EA), and calculate from the light integrals as follows:

$$EA = 100 \times \frac{Light Integral Tube B - Light Integral Tube A}{Light Integral Tube C - Light Integral Tube A .}$$

In this manner the EA is calculated and the decision of whether a patient is endotoxemic or not may be based on a cutoff value of range, i.e. >35 EA, an indicator of clinically significant endotoxemia.

Further parameters are available from the three-tube assay results as pertains to the stage of sepsis. Responsiveness (R) of the patients white blood cells, a measure of the maximal ability of the white blood cell to bind and respond to opsonized immunocomplexes as defined above, is calculated as follows:

$$R = 1 - [ \frac{Light Integral Tube A}{Light Integral Tube C} ] .$$

Furthermore, a measure of the level of white blood cell activation and cell number (CLₘₐₓ) may be measured as the peak luminometer count rate of tube A during the course of the assay. The maximum oxidant production of neutrophils, as measured by CLmax, is a measure of the ability of the white blood cell to respond to programmed opsonic challenge.

The following data in Table 1 is generated from the experiment. Explanations for the calculations of B-A, C-A, EA, and Responsiveness are provided above.

TABLE 1

| | | | | | | | Res- |
|---|---|---|---|---|---|---|---|
| | | | | | | | pon- |
| | Light Integral | | | | | | sive- |
| Sample | Tube A | Tube B | Tube C | B − A | C − A | EA | ness |
| 1 | 0.054 | 0.122 | 0.154 | 0.068 | 0.099 | 68 | 65 |
| 2 | 0.045 | 0.067 | 0.119 | 0.022 | 0.074 | 30 | 62 |
| 3 | 0.047 | 0.077 | 0.096 | 0.030 | 0.049 | 62 | 51 |
| 4 | 0.095 | 0.186 | 0.180 | 0.092 | 0.085 | 107 | 47 |
| 5 | 0.096 | 0.202 | 0.269 | 0.106 | 0.173 | 61 | 64 |
| 6 | 0.068 | 0.124 | 0.128 | 0.056 | 0.060 | 93 | 47 |
| 7 | 0.054 | 0.122 | 0.154 | 0.068 | 0.099 | 68 | 65 |
| 8 | 0.031 | 0.040 | 0.137 | 0.009 | 0.105 | 8 | 77 |
| 9 | 0.033 | 0.083 | 0.141 | 0.050 | 0.107 | 46 | 76 |
| 10 | 0.292 | 0.711 | 1.112 | 0.419 | 0.820 | 51 | 74 |
| 11 | 0.074 | 0.126 | 0.251 | 0.053 | 0.177 | 29 | 71 |
| 12 | 0.038 | 0.105 | 0.174 | 0.067 | 0.136 | 49 | 78 |
| 13 | 0.266 | 0.828 | 1.882 | 0.562 | 1.616 | 34 | 86 |
| 14 | 0.612 | 1.552 | 1.442 | 0.940 | 0.830 | 113 | 58 |
| 15 | 0.290 | 0.412 | 0.692 | 0.122 | 0.401 | 30 | 58 |
| 16 | 0.042 | 0.073 | 0.235 | 0.031 | 0.193 | 16 | 82 |
| 17 | 0.231 | 0.395 | 0.589 | 0.164 | 0.358 | 46 | 61 |
| 18 | 0.047 | 0.285 | 0.965 | 0.238 | 0.918 | 26 | 95 |

While the invention has been described and illustrated herein by references to various specific material, procedures and examples, it is understood that the invention is not restricted to the particular material combinations of material, and procedures selected for that purpose. Numerous variations of such details can be implied as will be appreciated by those skilled in the art.

Numerous citations are referred to in the Specification herein, all of which are incorporated herein in their entireties. Furthermore, this application herein incorporates in their entireties the following documents:

i) U.S. patent application Ser. No. 09/585,582 which is a continuation-in-part of application Ser. No. 09/353,189, filed Jul. 14, 1999; and a continuation-in-part of Ser. No. 09/457,465, filed Dec. 8, 1999, which is a continuation of Ser. No. 08/991,230, filed Dec. 16, 1997, now abandoned; both of which are a continuations-in-part of Ser. No. 08/552,145, filed Nov. 2, 1995; now U.S. Pat. No. 5,804,370; which is a continuation-in-part of Ser. No. 08/516,204, filed Aug. 17, 1995, abandoned; which is a continuation-in-part of Ser. No. 08/257,627, filed Jun. 8, 1994, abandoned; and

ii) U.S. patent application Ser. No. 09/961,889, which is a continuation-in-part of application Ser. No. 08/552,145, filed Nov. 2, 1995, now U.S. Pat. No. 5,804,370, which is a continuation-in-part of application Ser. No. 08/516,204, filed Aug. 17, 1995, abandoned, which is a continuation of application Ser. No. 08/257,627, filed Jun. 8, 1994, abandoned.

What is claimed is:

1. A method for measuring the amount of a preselected analyte in a sample comprising:
(a) forming an immunological complex between the analyte and an antibody thereto;
(b) reacting the complex with an oxidant-producing phagocytic cell or extract thereof; and
(c) measuring the amount of oxidant produced by said phagocytic cells as an indicator of the presence or absence of said analyte in said sample.

2. The method of claim 1 wherein said sample is a bodily fluid.

3. The method of claim 2 wherein said bodily fluid is whole blood.

4. The method of claim 2 wherein said oxidant-producing phagocytic cells are present in the sample of bodily fluid.

5. The method of claim 1 wherein an activator is included in step (b).

6. The method of claim 5 wherein said activator is selected from the group consisting of zymosan, latex particles, phorbol ester, fMLP, opsonized zymosan, opsonized latex particles, complement and any combination thereof.

7. The method of claim 1 wherein said analyte is indicative of the extent of infection or sepsis.

8. A method for measuring the amount of a preselected analyte in a sample comprising:
a. forming an immunocomplex between said preselected analyte and an antibody thereto;
b. reacting said immunocomplex with an oxidant-producing phagocytic cell in the presence of an activator; and
c. measuring the amount of oxidant produced as compared with that produced by a maximal amount of immunocomplexes between a second analyte and an antibody thereto in the presence of said activator as an indicator of the amount of said preselected analyte in said sample.

9. The method of claim 8 wherein said sample is a bodily fluid.

10. The method of claim 9 wherein said oxidant-producing phagocytic cells are present in the sample of bodily fluid.

11. The method of claim 9 wherein said bodily fluid is whole blood.

12. The method of claim 8 wherein said activator is selected from the group consisting of zymosan, latex particles, phorbol ester, fMLP, opsonized zymosan, opsonized latex particles, complement and any combination thereof.

13. The method of claim 8 wherein said preselected analyte is indicative of the of extent infection or sepsis.

14. The method of claim 8 wherein said second analyte is the same as the preselected analyte.

15. A method for detecting in sample of a bodily fluid a preselected analyte indicative of the extent of infection or sepsis which comprises:
a. forming an immunocomplex between said analyte and an antibody thereto;
b. reacting said immunocomplex with an oxidant-producing phagocytic cell in the presence of an activator; and
c. measuring the amount of oxidant produced as compared with that produced by a maximal amount of immunocomplexes between a second analyte and an antibody thereto in the presence of said activator as an indicator of the amount of said preselected analyte in said sample of said bodily fluid.

16. The method of claim 15 wherein said bodily fluid is whole blood.

17. The method of claim 15 wherein said oxidant-producing phagocytic cells are present in the sample of bodily fluid.

18. The method of claim 15 wherein said activator is selected from the group consisting of zymosan, latex particles, phorbol ester, FMLP, opsonized zymosan, opsonized latex particles, complement and any combination thereof.

19. The method of claim 15 wherein said preselected analyte is selected from the group consisting of Gram-positive bacteria, Gram-negative bacteria, a fungus, a virus, a protist, a Gram-positive cell wall constituent, Gram-negative endotoxin (lipopolysaccharide), lipid A, and an inflammatory mediator.

20. The method of claim 15 wherein said second analyte is the same as the preselected analyte.
